(19) United States
(12) Patent Application Publication
(10) Pub. No.: US 2018/0206897 A1
(43) Pub. Date: Jul. 26, 2018

(54) COMPRESSION SCREW SYSTEMS

(71) Applicant: ARTHREX, INC., Naples, FL (US)

(72) Inventor: Matthew Palmer, Medford, MA (US)

(21) Appl. No.: 15/879,997

(22) Filed: Jan. 25, 2018

Related U.S. Application Data
(60) Provisional application No. 62/450,273, filed on Jan. 25, 2017.

(51) Int. Cl. A61B 17/86; A61B 17/72; A61B 17/88

(52) U.S. Cl. CPC A61B 17/863; A61B 17/88; A61B 17/7225; A61B 17/864

(57) ABSTRACT

This disclosure details shape memory alloy compression screw systems and associated methods. The proposed compression screw systems and methods allow surgeons to create adequate compression across fracture sites or across joints.

### CROSS-REFERENCE TO RELATED APPLICATIONS

This disclosure claims priority to U.S. Provisional Application No. 62/450,273, which was filed on Jan. 25, 2017, the entire disclosure of which is hereby incorporated by reference herein.

### BACKGROUND

This disclosure relates to compression screw systems and methods. The compression screw systems may include shape memory alloys for imparting compression across two or more bone fragments.

Broken bones are commonly rejoined in the field of orthopedic surgery. The success of the bone rejoinder procedure often depends on the successful re-approximation of the bone fragments and on the amount of compression achieved between the bone fragments. If the surgeon is unable to bring the bone fragments into close proximity, a gap will exist between the bone fragments and the bone tissue will need to fill that gap before complete healing can take place.

Broken bones can be rejoined using screws, staples, plates, pins, intramedullary devices, and other fixation devices. However, known fracture fixation devices do not always succeed in bringing the bone fragments into close proximity and generating a compressive load between the bone fragments. Thus, improvements are desirable in this field of technology.

### SUMMARY

This disclosure details shape memory alloy compression screw systems and associated methods. The proposed systems and methods allow surgeons to create adequate compression across fracture sites or across joints. Compression can be maintained as the bones relax and remodel around the compression screw systems.

A method for treating a fracture or a joint includes, inter alia, inserting a compression screw system across a fracture of a bone or across a joint. The internal retaining pin is inserted in a cannulation of a screw body of the compression screw system. The screw body is in a reversibly stretched position and made of a shape memory alloy. The internal retaining pin is partially backed out without completely removing the internal retaining pin from the cannulation. Partially backing out of the internal retaining pin causes the screw body to attempt to return to an unstretched position, thereby creating a compressive force across the fracture or across the joint.

Another method for treating a fracture or a joint includes, inter alia, inserting a compression screw system across a fracture line between a first bone fragment and a second bone fragment or across a joint to impart a compressive force across the fracture line or across the joint. The compression screw system includes a first metallic piece having a proximal external screw thread with a first thread pitch, a second metallic piece having a distal external screw thread with a second thread pitch that is greater than the first thread pitch, and a shape memory alloy tie rod connecting the first metallic piece and the second metallic piece. A pitch differential between the proximal external screw thread and the distal external screw thread causes the distal external screw thread to advance into the first bone fragment and the second bone fragment faster than the proximal external screw thread, thereby stretching the shape memory alloy tie rod from an unstretched position to a stretched position. After the compression screw system is inserted fully across the fracture line, the shape memory alloy tie rod attempts to foreshorten to the unstretched position, thereby imparting an additional compressive force across the fracture line or across the joint.

A compression screw system includes, inter alia, a two-piece metallic section including a) a first piece having an unthreaded central shaft and a proximal external screw thread and b) a second piece having a distal external screw thread. The unthreaded central shaft is slidably received within the second piece. A shape memory alloy tie rod extends inside a first cannulation of the first piece and a second cannulation of the second piece. The shape memory alloy tie rod is axially stretchable between an unstretched position and a stretched position as the proximal external screw thread and the distal external screw thread engage bone.

### BRIEF DESCRIPTION OF THE DRAWINGS

FIG. 1 illustrates a compression screw system according to a first embodiment of this disclosure.

FIG. 2 illustrates a screw body of the compression screw system of FIG. 1.

FIGS. 3 and 4 illustrate an internal retaining pin of the compression screw system of FIG. 1.

FIGS. 5 and 6 schematically illustrate reversibly straining and unstraining a portion of the compression screw system of FIG. 1.

FIG. 7 is a graphical depiction of the behavior of the screw body of the compression screw system of FIG. 1 during strain loading and unloading.

FIG. 8 illustrates a compression screw system according to a second embodiment of this disclosure.

FIG. 9 is a cross-sectional view of the compression screw system of FIG. 8.

FIGS. 10 and 11 illustrate additional features of the compression screw system of FIG. 8.

FIGS. 12A, 12B, 12C, 12D, 12E, and 12F schematically illustrate a method of treating a fracture using the compression screw system of FIGS. 8-11.

### DETAILED DESCRIPTION

This disclosure describes exemplary shape memory alloy compression screw systems. In an embodiment, the compression screw systems include one or more components made of Nitinol (NiTi) or a similar shape memory alloy. The proposed designs allow surgeons to create adequate compression across fracture sites or across joints. Compression can be maintained as the bones relax and remodel around the compression screw systems, thereby improving healing.

A method for treating a fracture or a joint includes, inter alia, inserting a compression screw system across a fracture of a bone or across a joint. The internal retaining pin is inserted in a cannulation of a screw body of the compression screw system. The screw body is in a reversibly stretched position and made of a shape memory alloy. The internal retaining pin is partially backed out without completely removing the internal retaining pin from the cannulation. Partially backing out of the internal retaining pin causes the screw body to attempt to return to an unstretched position, thereby creating a compressive force across the fracture or across the joint.

In a further embodiment, a stretched position of a screw body of a compression screw system is axially stretched relative to an unstretched position.

In a further embodiment, a screw body of a compression screw system includes proximal threads, distal threads, and a central shaft extending between the proximal threads and the distal threads.

In a further embodiment, reversibly stretching a screw body of a compression screw system includes stretching a central shaft of the screw body.

In a further embodiment, a shape memory alloy of a screw body of a compression screw system includes Nitinol.

In a further embodiment, an internal retaining pin of a compression screw system is made of a biocompatible alloy.

In a further embodiment, an internal retaining pin of a compression screw system abuts a shoulder located within a portion of a cannulation of a screw body when the compression screw system is inserted across a fracture or across a joint.

In a further embodiment, partially backing out an internal retaining pin of a compression screw system includes turning the internal retaining pin until a gap extends between a distal tip of the internal retaining pin and a shoulder of a screw body cannulation.

In a further embodiment, an internal retaining pin of a compression screw system is unthreaded.

In a further embodiment, a screw body of a compression screw system includes a monolithic design.

Another method for treating a fracture or a joint includes, inter alia, inserting a compression screw system across a fracture line between a first bone fragment and a second bone fragment or across a joint to impart a compressive force across the fracture line or across the joint. The compression screw system includes a first metallic piece having a proximal external screw thread with a first thread pitch, a second metallic piece having a distal external screw thread with a second thread pitch that is greater than the first thread pitch, and a shape memory alloy tie rod connecting the first metallic piece and the second metallic piece. A pitch differential between the proximal external screw thread and the distal external screw thread causes the distal external screw thread to advance into the first bone fragment and the second bone fragment faster than the proximal external screw thread, thereby stretching the shape memory alloy tie rod from an unstretched position to a stretched position. After the compression screw system is inserted fully across the fracture line, the shape memory alloy tie rod attempts to foreshorten to the unstretched position, thereby imparting an additional compressive force across the fracture line or across the joint.

In a further embodiment, a k-wire is inserted across a fracture line prior to inserting a compression screw system.

In a further embodiment, a compression screw system is inserted over a k-wire.

In a further embodiment, a first metallic piece of a compression screw system includes an unthreaded central shaft that is received is telescoping engagement with a second metallic piece.

In a further embodiment, a shape memory alloy tie rod of a compression screw system is received within a cannulation formed inside each of a first metallic piece and a second metallic piece.

A compression screw system includes, inter alia, a two-piece metallic section including a) a first piece having an unthreaded central shaft and a proximal external screw thread and b) a second piece having a distal external screw thread. The unthreaded central shaft is slidably received within the second piece. A shape memory alloy tie rod extends inside a first cannulation of the first piece and a second cannulation of the second piece. The shape memory alloy tie rod is axially stretchable between an unstretched position and a stretched position as the proximal external screw thread and the distal external screw thread engage bone.

In a further embodiment, a shape memory alloy tie rod of a compression screw system is made of Nitinol.

In a further embodiment, a first piece of a metallic section of a compression screw system is received within a second piece in a telescoping manner.

In a further embodiment, a proximal end of a shape memory alloy tie rod of a compression screw system abuts a shoulder of a first piece and a distal end of the shape memory alloy tie rod threads into a threaded portion of a second piece.

In a further embodiment, a proximal external screw thread of a compression screw system includes a first thread pitch and a distal external screw thread includes a second thread pitch that is greater than the first thread pitch.

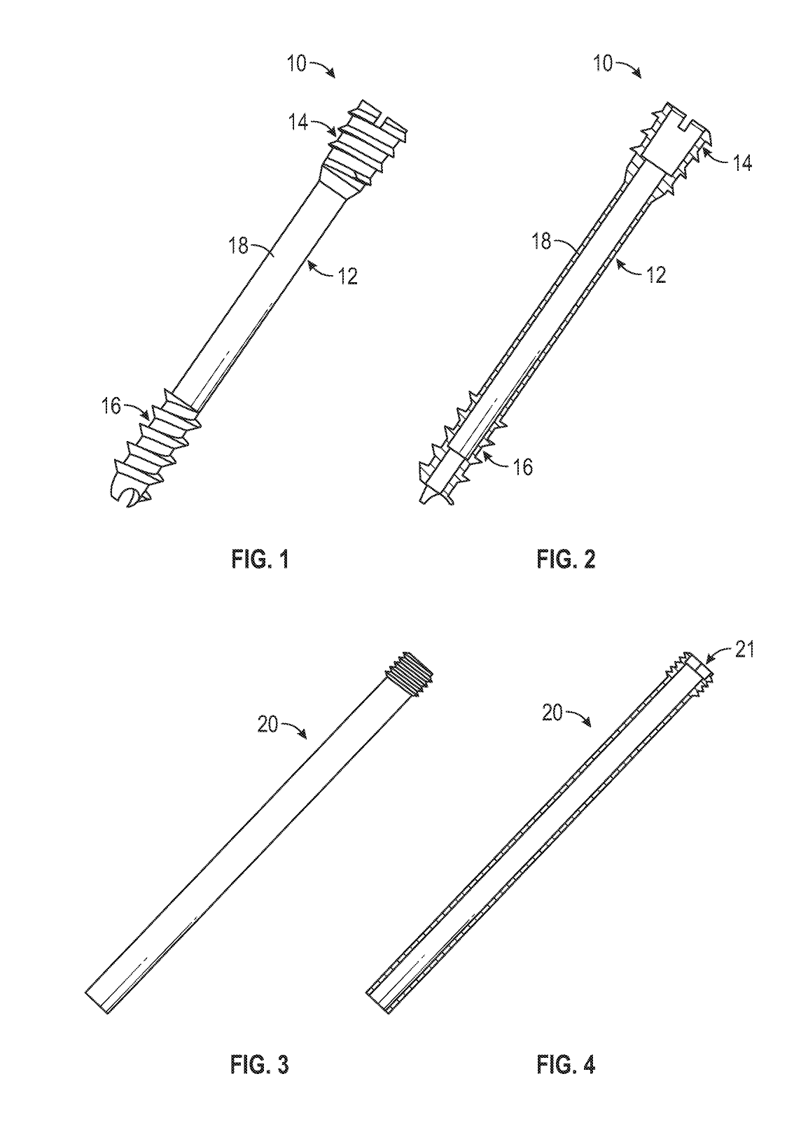

FIGS. 1-6 illustrate an exemplary compression screw system 10. In an embodiment, the compression screw system 10 includes components made of Nitinol. The compression screw system 10 includes a screw body 12 that is fully cannulated and has proximal threads 14 and distal threads 16 connected by a central shaft 18. The central shaft 18 may be threadless. In this embodiment, the screw body 12 includes a single piece and therefore embodies a monolithic design. The screw body 12 may be made of or include Nitinol.

The central shaft 18 can be reversibly strained. By changing the cross-sectional area (ID and OD) of the central shaft 18, the amount of force required to strain the screw body 12 can be altered. Additionally, by changing the cross-sectional area of the central shaft 18 region, the amount of force generated as the screw body 12 recovers strain can be controlled. This force is equivalent to the compression between two bone fragments.

The screw body 12 can be reversibly strained and then an internal retaining pin 20 (also cannulated, see FIGS. 3-4) can be threaded into the screw body 12 to maintain the screw body 12 in its elongated state. After implantation of the screw body 12, the internal retaining pin 20 can be unthreaded, thereby allowing the screw body 12 to recover the stored strain.

The internal retaining pin 20 may include a drive feature 21 (see FIG. 4) for turning the internal retaining pin 20 within the cannulated screw body 12. The drive feature 21 can be a standard screw drive feature such as a drive slot, a Philips (cruciform) drive configuration, a hex or hexalobe recess, or any other engagement feature.

The internal retaining pin 20 can be made from various biocompatible alloys. Non-limiting examples of suitable biocompatible alloys include stainless steel, titanium, L605, and MP35N.

For relatively large diameter screws, it is not possible to reduce the cross-sectional area sufficiently (to reduce the compressive force generated during strain recovery) and still maintain adequate screw mechanical properties (bending and fatigue). To counter this, the internal retaining pin 20 may only be partially backed out (to allow for strain recovery). The amount the internal retaining pin 20 is backed out is at the discretion of the surgeon, and thus the amount of compression can be tailored to each specific application. In embodiments in which the internal retaining pin 20 is backed out only partially and thus retained within the screw body 12 after implantation, the internal retaining pin 20 acts as a backing plate for reinforcing the screw body 12 and increasing mechanical strength. In other embodiments, the cannulated internal retaining pin 20 can be fully removed and a non-cannulated internal pin can be threaded into the screw to provide even more superior mechanical properties.

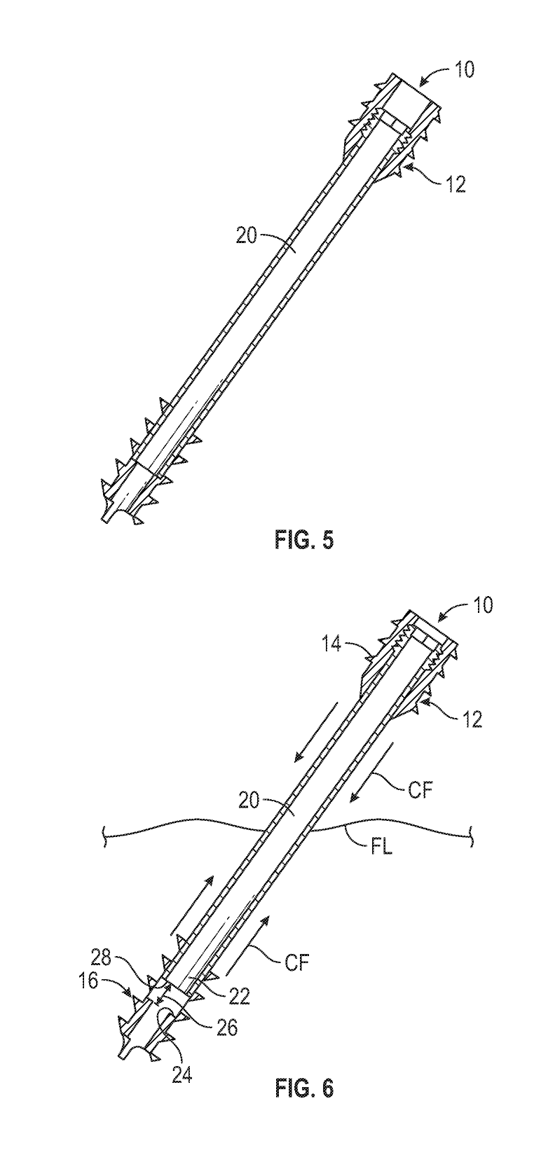

FIGS. 5 and 6 schematically illustrate reversibly straining and unstraining the compression screw system 10 in order to achieve a desired amount of compression across a fracture line FL (see FIG. 6). The screw body 12 is first stretched, and then the internal retaining pin 20 is inserted into the cannulation 22 of the screw body 12. The internal retaining pin 20 may be inserted until it abuts against a shoulder 24 located within the portion of the cannulation 22 that may extend inside the distal thread 16 or may terminate prior to the distal thread 16.

The compression screw system 10 is then inserted into bone across the fracture line FL. Once positioned as desired, the surgeon may partially back out the internal retaining pin 20 such that a gap 26 extends between a distal tip 28 of the internal retaining pin 20 and the shoulder 24 of the cannulation 22. The amount the internal retaining pin 20 is backed up may be calibrated or could be up to the discretion of the surgeon. As the internal retaining pin 20 is partially backed out, the screw body 12 attempts to recover the stored strain, thereby creating a compressive force (see arrows CF) that causes the proximal threads 14 and distal threads 16 to move toward one another.

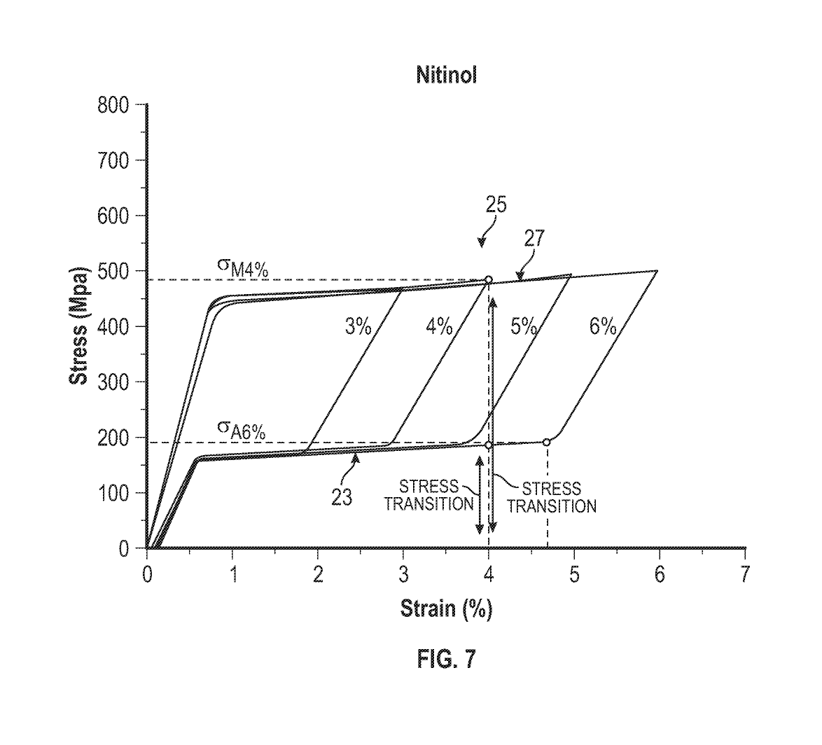

FIG. 7 graphically depicts the behavior of the screw body 12 of the compression screw system 10 during strain loading and unloading. As depicted by FIG. 7, the amount of stress imposed on the bone by the compression screw system 10 differs significantly between: (i) a first situation where the screw body 12 (i.e., the Nitinol device) is stressed (e.g., to a 6% strain), then “backed off” to the lower plateau 23 of the mechanical hysteresis curve 25, then further “backed off” along the lower plateau 23 to a given level of strain (e.g., to a 4% strain), then constrained at that given level of strain (e.g., a 4% strain), and then released into the bone at that given level of strain (e.g., a 4% strain)—whereupon the maximum stress generated by the screw body 12 never rises above the lower plateau 23 of the mechanical hysteresis curve (i.e., the maximum stress generated by the screw body 12 never rises above σ_{A6%}; and (ii) a second situation where the screw body 12 is stressed to that same given level of strain (e.g., 4% strain), then constrained at that same given level of strain (e.g., 4% strain), and then released into the bone at that same given level of strain (e.g., 4% strain)—whereupon the maximum stress generated by the screw body 12 starts at the upper plateau 27 of the mechanical hysteresis curve 25 (i.e., the maximum stress generated by the screw body 12 reaches σ_{M4%}). In other words, the stress transition produced in the aforementioned first situation (i.e., where the screw body 12 is unloaded to the lower plateau 23 of the mechanical hysteresis curve 25 prior to implantation) is significantly lower than the stress transition produced in the aforementioned second situation (i.e., where the screw body 12 is fully unloaded after implantation). Thus, the same amount of strain recovery (e.g., 4%) can exhibit different stress levels depending on whether the device begins unloading at the upper plateau 27 or the lower plateau 23.

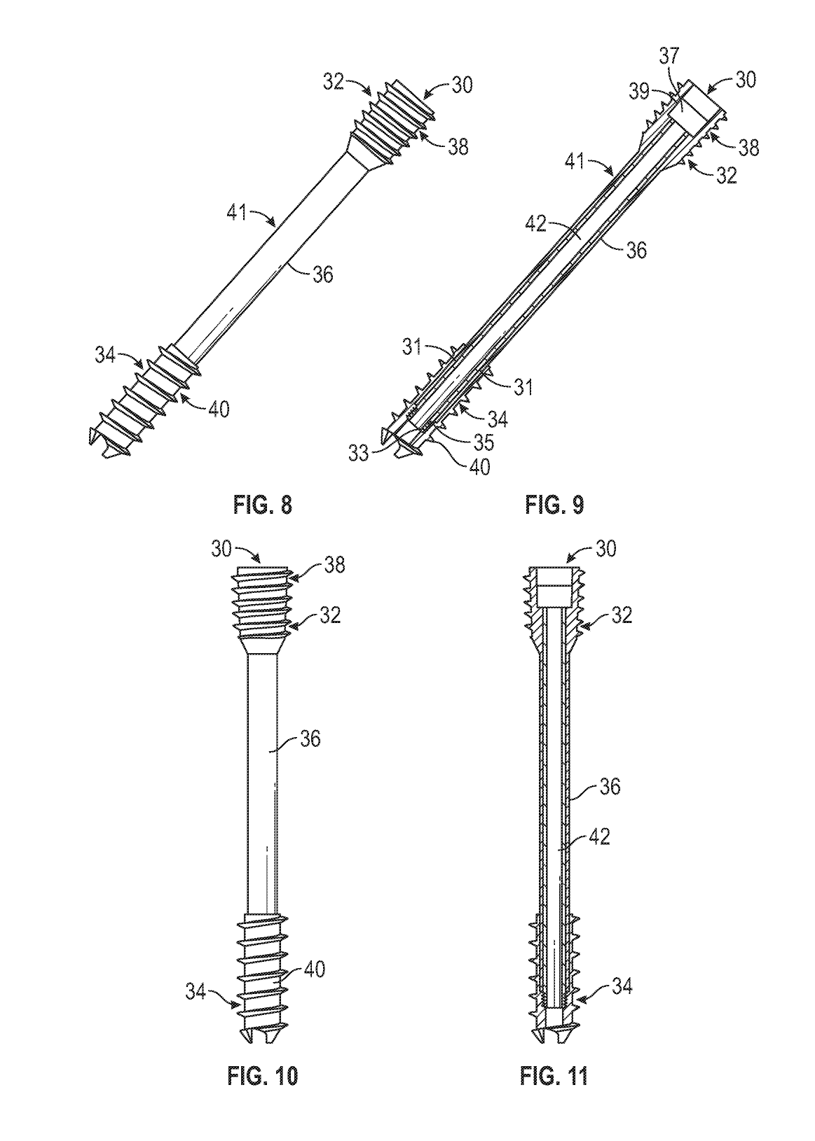

FIGS. 8-11 illustrate another exemplary compression screw system 30. In this embodiment, the compression screw system 30 includes a two-piece metallic section 41 having a first piece 32 and a second piece 34. The metallic section 41 may be made of titanium, stainless steel, or any other biocompatible material. The first and second pieces 32, 34 of the metallic section 41 may slide within each other, such as in a telescoping manner. The first piece 32 is fully cannulated and includes an unthreaded central shaft 36 with proximal external screw threads 38. The first piece 32 may additionally include flats 31 machined at the distal end.

The second piece 34 is also fully cannulated and has distal external screw threads 40. The proximal end of the cannulation is machined so as to allow the flats from the first piece 32 to slide within the second piece 34. The first and second pieces 32, 34 slide within each other but cannot rotate relative to each other. The distal end of the internal cannulation of the second piece 34 includes a threaded portion 33.

The first and second pieces 32, 34 of the metallic section 41 may be connected by a reversibly strainable shape memory alloy tie rod 42. In an embodiment, the shape memory alloy tie rod 42 is made of Nitinol. The shape memory alloy tie rod 42 is also fully cannulated. It can be passed through the first piece 32 of the metallic section 41 and includes a threaded portion 35 can be threaded into the threaded portion 33 of the second piece 34. A proximal end 37 of the shape memory alloy tie rod 42 may abut a shoulder 39 of the first piece 32.

The proximal external screw threads 38 are finer (i.e., more threads per inch) than the distal external screw threads 40 (i.e., fewer threads per inch). In other words, the distal external screw threads 40 have a greater thread pitch than the proximal external screw threads 38. Thus, with each rotation the distal external screw threads 40 (bottom piece of the screw) want to advance into the bone more than the proximal external screw threads 38 (top piece of the screw). This thread pitch differential reversibly stretches the shape memory alloy tie rod 42. After the screw is fully implanted, the shape memory alloy tie rod 42 wants to recover the strain and thus maintains compression between the bone fragments.

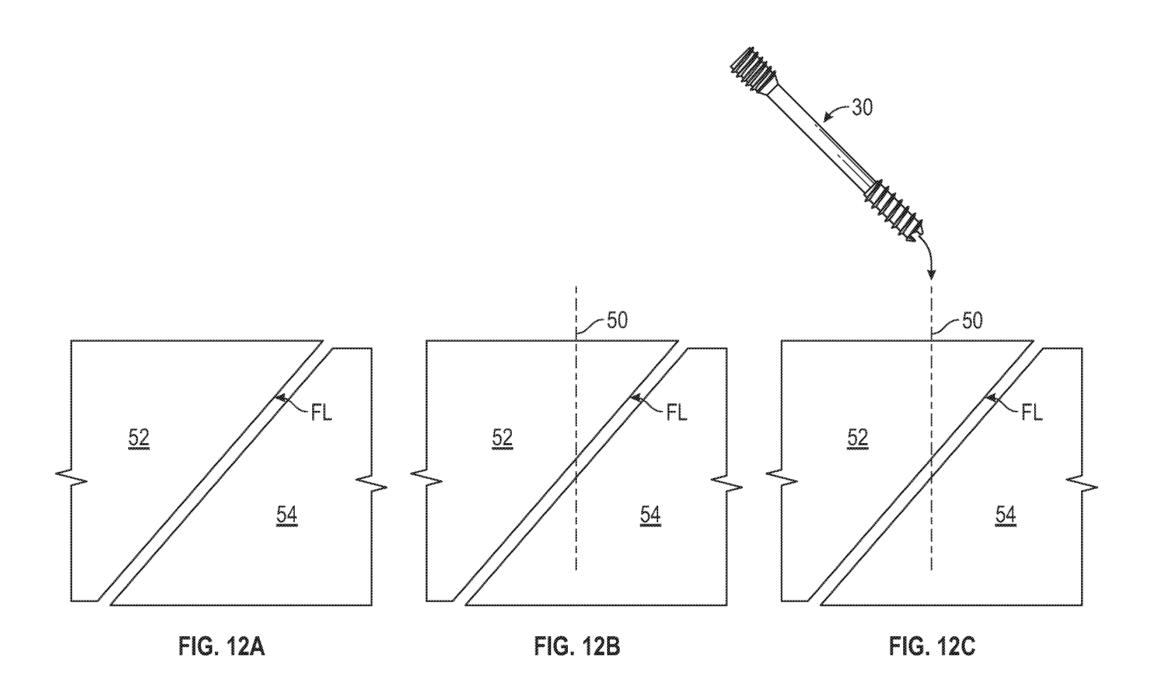

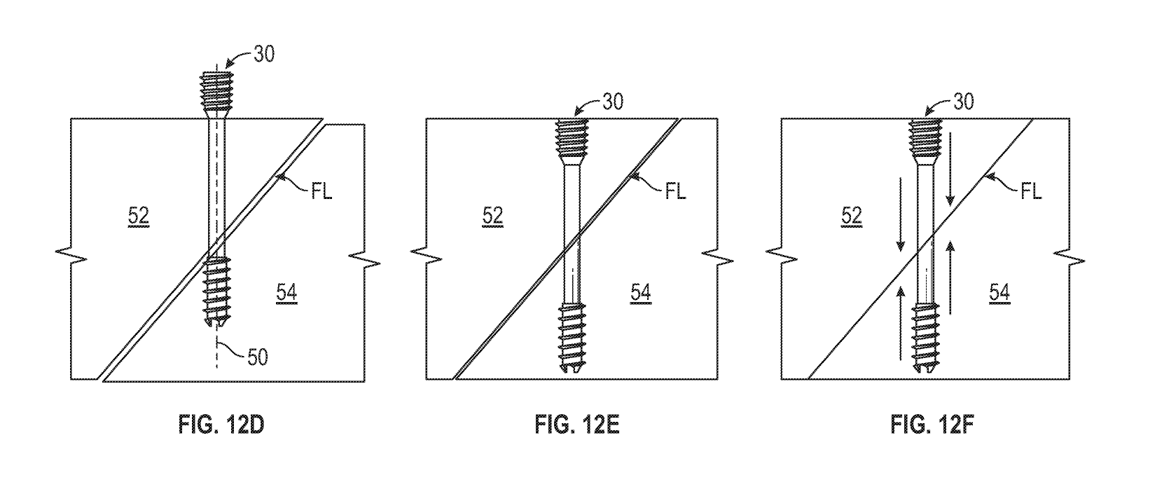

FIGS. 12A-12F schematically illustrates a method of treating a fracture using the compression screw system 30. In an embodiment, a k-wire 50 is inserted across a fracture line FL to provisionally stabilize bone fragments 52, 54 (see FIG. 12B). The compression screw system 30 may then be inserted over the k-wire 50 and threaded into the bone fragments 52, 54 so that the compression screw system 30 extends across the fracture line FL (see FIGS. 12C and 12D). The differential pitch between the proximal external screw threads 38 and the distal external screw threads 40 creates compression across fracture line FL and reduces the fracture (see FIGS. 12D and 12E). As the compression screw system 30 is thoroughly countersunk into the bone, the pitch differential between the proximal external screw threads 38 and the distal external screw threads 40 generates sufficient axial tension in the unthreaded central shaft 36 to reversibly stretch the unthreaded central shaft 36 (via the shape memory alloy tie rod 42) by up to about 8%. With each rotation of the compression screw system 30, the distal external screw threads 40 advance into the bone faster than the proximal external screw threads 38. This thread differential (and hence thread advancement differential) creates the axial tension in the unthreaded central shaft 36 of the compression screw system 30. The K-wire 50 may then be removed.

With the implantation of compression screw system 30 complete, the unthreaded central shaft 36 will attempt to foreshorten to its pre-strained (i.e., pre-stretched) condition (see FIG. 12F). The foreshortening of the unthreaded central shaft 36 of the compression screw system 30 generates an additional compressive load across the fracture line FL, thereby enhancing healing.

Although the different non-limiting embodiments are illustrated as having specific components or steps, the embodiments of this disclosure are not limited to those particular combinations. It is possible to use some of the components or features from any of the non-limiting embodiments in combination with features or components from any of the other non-limiting embodiments.

It should be understood that like reference numerals identify corresponding or similar elements throughout the several drawings. It should further be understood that although a particular component arrangement is disclosed and illustrated in these exemplary embodiments, other arrangements could also benefit from the teachings of this disclosure.

The foregoing description shall be interpreted as illustrative and not in any limiting sense. A worker of ordinary skill in the art would understand that certain modifications could come within the scope of this disclosure. For these reasons, the following claims should be studied to determine the true scope and content of this disclosure.

What is claimed is:

1. A method for treating a fracture or a joint, comprising:
inserting a compression screw system across a fracture of a bone or across a joint, wherein an internal retaining pin is inserted in a cannulation of a screw body of the compression screw system, wherein the screw body is in a reversibly stretched position and made of a shape memory alloy; and
partially backing out the internal retaining pin without completely removing the internal retaining pin from the cannulation;
wherein the partially backing out of the internal retaining pin causes the screw body to attempt to return to an unstretched position, thereby creating a compressive force across the fracture or across the joint.

2. The method as recited in claim 1, wherein the stretched position is axially stretched relative to the unstretched position.

3. The method as recited in claim 1, wherein the screw body includes proximal threads, distal threads, and a central shaft extending between the proximal threads and the distal threads.

4. The method as recited in claim 3, wherein reversibly stretching the screw body includes stretching the central shaft.

5. The method as recited in claim 1, wherein the shape memory alloy includes Nitinol.

6. The method as recited in claim 1, wherein the internal retaining pin is made of a biocompatible alloy.

7. The method as recited in claim 1, wherein the internal retaining pin abuts a shoulder located within a portion of the cannulation of the screw body when the compression screw system is inserted across the fracture or across the joint.

8. The method as recited in claim 1, wherein the partially backing out of the internal retaining pin includes turning the internal retaining pin until a gap extends between a distal tip of the internal retaining pin and a shoulder of the cannulation.

9. The method as recited in claim 1, wherein the internal retaining pin is unthreaded.

10. The method as recited in claim 1, wherein the screw body includes a monolithic design.

11. A method for treating a fracture or a joint, comprising:
inserting a compression screw system across a fracture line between a first bone fragment and a second bone fragment or across a joint to impart a compressive force across the fracture line or across the joint,
wherein the compression screw system includes a first metallic piece having a proximal external screw thread with a first thread pitch, a second metallic piece having a distal external screw thread with a second thread pitch that is greater than the first thread pitch, and a shape memory alloy tie rod connecting the first metallic piece and the second metallic piece,
wherein a pitch differential between the proximal external screw thread and the distal external screw thread causes the distal external screw thread to advance into the first bone fragment and the second bone fragment faster than the proximal external screw thread, thereby stretching the shape memory alloy tie rod from an unstretched position to a stretched position, and
wherein, after the compression screw system is inserted fully across the fracture line, the shape memory alloy tie rod attempts to foreshorten to the unstretched position, thereby imparting an additional compressive force across the fracture line or across the joint.

12. The method as recited in claim 11, comprising inserting a k-wire across the fracture line prior to inserting the compression screw system.

13. The method as recited in claim 12, wherein the compression screw system is inserted over the k-wire.

14. The method as recited in claim 11, wherein the first metallic piece includes an unthreaded central shaft that is received is telescoping engagement with the second metallic piece.

15. The method as recited in claim 11, wherein the shape memory alloy tie rod is received within a cannulation formed inside each of the first metallic piece and the second metallic piece.

16. A compression screw system, comprising:
a two-piece metallic section including a) a first piece having an unthreaded central shaft and a proximal external screw thread and b) a second piece having a distal external screw thread, wherein the unthreaded central shaft is slidably received within the second piece; and
a shape memory alloy tie rod extending inside a first cannulation of the first piece and a second cannulation of the second piece;
wherein the shape memory alloy tie rod is axially stretchable between an unstretched position and a stretched position as the proximal external screw thread and the distal external screw thread engage bone.

17. The system as recited in claim 16, wherein the shape memory alloy tie rod is made of Nitinol.

18. The system as recited in claim 16, wherein the first piece is received within the second piece in a telescoping manner.

19. The system as recited in claim 16, wherein a proximal end of the shape memory alloy tie rod abuts a shoulder of the first piece and a distal end of the shape memory alloy tie rod threads into a threaded portion of the second piece.

20. The system as recited in claim 16, wherein the proximal external screw thread includes a first thread pitch and the distal external screw thread includes a second thread pitch that is greater than the first thread pitch.
